(19) United States
(12) Patent Application Publication
(10) Pub. No.: US 2026/0346054 A1
(43) Pub. Date: Nov. 5, 2026

(54) SYSTEMS AND METHODS FOR BIOFILM REMOVAL

(71) Applicant: Orthopaedic Technology Solutions, LLC, Matthews, NC (US)

(72) Inventors: Matthew B. SHIRLEY, Heber City, UT (US); Thomas K. FEHRING, Charlotte, NC (US); Jesse E. OTERO, Matthews, NC (US)

(21) Appl. No.: 19/663,592

(22) Filed: Apr. 30, 2026

Related U.S. Application Data
(60) Provisional application No. 63/895,864, filed on Oct. 8, 2025.
(60) Provisional application No. 63/829,524, filed on Jun. 24, 2025.
(60) Provisional application No. 63/798,266, filed on May 1, 2025.

(51) Int. Cl. A61N 7/00; A61F 2/30

(52) U.S. Cl. CPC A61N 7/00; A61F 2/30; A61F 2002/30719

(57) ABSTRACT

Embodiments herein provide systems and devices for removing biofilms at treatment sites. Certain embodiments describe a sheath that is configured to be removably attached to an ultrasonic probe. The sheath includes a body configured to define an internal cavity for receiving and enclosing at least the ultrasonic assembly of the probe, and a sheath head coupled at a distal end of the body. The sheath head is configured to function as a spacer to form an optimal stand-off distance between the ultrasonic assembly and a treatment site when the sheath head is pressed against the treatment site, and/or maintain a fluid within an internal volume of the sheath head when the sheath head is pressed against the treatment site to provide a fluid transmission medium between the ultrasonic assembly and the treatment site.

### CROSS-REFERENCE TO RELATED APPLICATIONS

This application claims benefit of United States Provisional Patent Application No. 63/798,266, filed May 1, 2025, United States Provisional Patent Application No. 63/829,524, filed Jun. 24, 2025, and United States Provisional Patent Application No. 63/895,864, filed Oct. 8, 2025. Each of the aforementioned related patent applications is herein incorporated by reference in its entirely.

### INTRODUCTION

Periprosthetic joint infection (PJI) is a serious and challenging complication associated with orthopedic joint replacement surgeries, including total hip, knee, and shoulder arthroplasties. PJIs are caused by microbial contamination of the implant and/or surrounding tissues and may occur intraoperatively, in the early postoperative period, or even years after implantation due to hematogenous spread. These infections can result in the loosening of implants, osteolysis, chronic pain, and other significant patient morbidities.

Traditional methods for managing PJI generally include a combination of surgical intervention and systemic antibiotic therapy. Among the most commonly utilized surgical strategies is “DAIR”—debridement, antibiotics, and implant retention—which is frequently considered in acute or early postoperative infections where the implant remains well-fixed and microbial biofilm formation is presumed to be limited. In a DAIR procedure, the infected joint is accessed surgically, infected soft tissue is debrided, and the joint is irrigated—often with high-pressure lavage—followed by the exchange of modular prosthetic components (e.g., polyethylene liners) while retaining fixed implant hardware. Systemic antibiotics are then administered to eradicate residual infection.

Other conventional techniques for acute infections include irrigation and debridement (I&D) with implant retention, which may be used in early infections where the implant remains stable and biofilm formation is minimal. These procedures typically involve aggressive lavage of the prosthesis site with sterile fluids, mechanical scraping of infected tissue, and replacement of modular implant components.

In cases of chronic infection, more invasive revision procedures may be required. These include reconstruction and debridement, which may refer to either one-stage or two-stage revision arthroplasty. In a one-stage revision, the infected prosthesis is removed, the joint is thoroughly debrided, and a new prosthesis is implanted during the same surgery. In a two-stage revision, after removal of the infected components and surgical debridement, a temporary antibiotic-loaded spacer is inserted. This is followed by a course of systemic antibiotics, with subsequent reimplantation of a new prosthesis after infection control is confirmed.

Despite these approaches, PJIs are notoriously difficult to eradicate due to the presence of microbial biofilms on implant surfaces and necrotic tissue, which can protect microorganisms from antibiotics and immune responses. As such, there remains a need for more effective systems and intraoperative methods for decontaminating the surgical site, removing biofilm, and facilitating eradication of infection without extensive tissue damage or multiple surgical interventions.

### SUMMARY

Embodiments of the present disclosure provide improved systems and methods for intraoperative removal and treatment of microbial biofilms, such as bacterial biofilms formed on prosthetic joints. Certain embodiments of the present disclosure further provide improved systems and methods for removing, or breaking off, bacteria from infected prosthesis for diagnostic purposes.

For example, some embodiments of the present disclosure provide systems and methods for generating a localized, fluid-mediated ultrasonic cavitation environment at a treatment site to disrupt, detach, and remove biological materials, including microbial biofilms, from surfaces. In such embodiments, the disclosed systems can utilize a sheath head configured to define and maintain a confined fluid volume between an ultrasonic emitter and the treatment site, thereby enhancing acoustic coupling and increasing cavitation density relative to non-confined fluid delivery approaches. This controlled cavitation environment enables targeted disruption of biofilms while reducing unintended energy dissipation and minimizing damage to adjacent tissue or structures. In some embodiments, the systems further enable controlled detachment and collection of biological material for diagnostic analysis.

In at least some embodiments, an ultrasonic system for removing a biofilm at a treatment site is provided, the ultrasonic system comprising: an ultrasonic probe, comprising: an ultrasonic assembly, comprising: a transducer configured to generate ultrasonic energy; and a horn in operable communication with the transducer and configured to receive, amplify, and focus the ultrasonic energy generated by the transducer; and a handpiece mechanically and electrically coupled to the ultrasonic assembly and configured to be held and manipulated by the user; a sheath configured to be removably attached to the ultrasonic probe, the sheath comprising: a body configured to define an internal cavity for receiving and enclosing at least the ultrasonic assembly; and a sheath head coupled at a distal end of the body, the sheath head configured to perform at least one of: function as a spacer to form an optimal stand-off distance between a distal end of the horn and a treatment site when the sheath head is pressed against the treatment site; or maintain a fluid within an internal volume of the sheath head when the sheath head is pressed against the treatment site to provide a fluid transmission medium between the distal end of the horn and the treatment site.

In at least some embodiments, a sheath for an ultrasonic probe is provided, the sheath comprising: a body configured to removably couple to an ultrasonic probe to enclose at least one of a horn or transducer of the ultrasonic probe; and a sheath head coupled to a distal end of the body, the sheath head configured to perform at least one of: function as a spacer to form an optimal stand-off distance between a distal end of the horn and a treatment site when the sheath head is pressed against the treatment site; or maintain a fluid within an internal volume of the sheath head when the sheath head is pressed against the treatment site to provide a fluid transmission medium between the distal end of the horn and the treatment site.

In some embodiments, the present disclosure provides methods for treating a surface, including biological tissue and implantable devices, using the ultrasonic systems described herein. In some embodiments, a method comprises positioning a sheath head against a treatment site such that the sheath head and the treatment site define a confined fluid volume. The method further comprises introducing a fluid into the confined fluid volume and delivering ultrasonic energy from a horn into the fluid, thereby generating cavitation within the confined fluid volume. The cavitation disrupts and detaches biological material, including microbial biofilm, from the treatment site. In some embodiments, the method is performed while the treatment site remains in situ within a patient. In some embodiments, the method further comprises removing detached material from the confined fluid volume, including via passive outflow or active suction. In some embodiments, the detached material is collected and analyzed to identify microbial content.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a schematic view of an example ultrasonic treatment system for use during orthopedic and/or other surgical procedures, according to at least some embodiments of the present disclosure.

FIG. 2 is a schematic and partially cross-sectional view of an example probe of the ultrasonic treatment system of FIG. 1, according to at least some embodiments of the present disclosure.

FIG. 3A is a perspective view of an example probe of the ultrasonic treatment system of FIG. 1, with an example sheath attached thereto, according to at least some embodiments of the present disclosure.

FIG. 3B is a cross-sectional view of the probe and sheath of FIG. 3A, according to at least some embodiments of the present disclosure.

FIG. 3C is a cross-sectional view of the sheath of FIG. 3A, according to at least some embodiments of the present disclosure.

FIG. 3D is an enlarged cross-sectional view of a sheath head of the sheath of FIG. 3A, according to at least some embodiments of the present disclosure.

FIG. 4A is a perspective view of the probe of FIG. 3A with another example sheath, according to at least some embodiments of the present disclosure.

FIG. 4B is an enlarged cross-sectional view of a sheath head of the sheath of FIG. 4A, according to at least some embodiments of the present disclosure.

FIG. 5A is a perspective view of the probe of FIG. 3A with another example sheath, according to at least some embodiments of the present disclosure.

FIG. 5B is an enlarged cross-sectional view of a sheath head of the sheath of FIG. 5A, according to at least some embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Periprosthetic joint infection (PJI) is a significant complication following orthopedic implant procedures, often requiring complex surgical intervention and prolonged antibiotic therapy. Conventional treatment strategies aim to manage infection by physically removing contaminated tissue, implants, and/or administering antimicrobial agents. However, microbial biofilms that form on implant surfaces and surrounding tissues can be highly resistant to antibiotics and immune responses, and can be difficult to eliminate with standard irrigation and/or mechanical techniques. As a result, such infections frequently persist or recur, sometimes requiring invasive revision surgeries that impose significant risks and recovery burdens on patients.

To address these limitations, the present application provides ultrasonic treatment systems to intraoperatively disrupt and remove microbial biofilms from implant surfaces and adjacent tissues during orthopedic surgery. The systems utilize high-frequency ultrasonic energy delivered via handheld probes specifically configured to enhance debridement efficacy while preserving surrounding healthy structures. This approach offers the potential to significantly improve intraoperative infection control and patient outcomes in both acute and chronic PJI cases.

In contrast to conventional ultrasonic systems in which acoustic energy propagates through unconfined or continuously flowing fluid, the present disclosure provides a confined fluid transmission medium defined at least in part by a sheath head of a probe sheath. The confined fluid volume increases local acoustic pressure amplitude and promotes formation of cavitation bubbles within a spatially controlled region adjacent to the treatment site. Without being bound by theory, confinement of the fluid within the sheath head is believed to limit volumetric expansion of the fluid during ultrasonic excitation, thereby increasing localized pressure oscillations and promoting formation of higher-density cavitation bubbles. Additionally, the confined geometry may reduce convective dissipation of acoustic energy, resulting in increased energy density at the treatment site relative to systems employing unconfined or continuously flowing fluid.

The sheath head geometry and the maintained stand-off distance between the distal end of the horn and the treatment site thus cooperate to define a localized cavitation zone. This configuration enhances the efficiency of ultrasonic energy transfer and reduces energy dissipation into surrounding fluid or tissue. In some embodiments, the stand-off distance is selected to approximate a resonance or near-resonance condition for the ultrasonic frequency used, thereby further increasing cavitation intensity within the confined fluid volume. The resulting localized cavitation field facilitates disruption of biofilm structures adhered to a surface while minimizing mechanical or thermal damage to adjacent tissue or structural components.

In some embodiments, maintaining a relatively quiescent or low-flow fluid environment within the confined volume promotes formation and collapse of cavitation bubbles at or near the treatment surface. Whereas, increased bulk fluid flow may transport cavitation bubbles away from the treatment region and reduce localized cavitation intensity. Similarly, reductions in ambient pressure within the confined volume may alter bubble dynamics and reduce the mechanical impact of cavitation events on the treatment surface. Accordingly, in some embodiments, the ultrasonic treatment systems herein are configured to maintain a substantially static or low-flow fluid environment within the confined volume by introducing fluid at a controlled rate and allowing passive egress of the fluid without active suction, thereby promoting stable cavitation and efficient energy transfer at the treatment site.

Additionally, conventional lavage techniques rely on bulk fluid flow for cleaning, whereas the present systems utilize localized cavitation to mechanically disrupt adherent material. These differences can result in improved removal of biofilm while reducing the need for high-pressure fluid delivery or aggressive mechanical intervention.

Examples will now be described relative to the Drawings.

Note that, as described herein, a “distal” end, side, or portion of a component refers to the end, side, or the portion that is closer to the patient’s body during the use thereof (e.g., at a far end away from the surgical device). On the other hand, a “proximal” end, side, or portion of the component refers to the end, side, or portion that is distanced further away from the patient’s body (e.g., is closer to the surgical device).

As used herein, “about,” when used in conjunction with a numerical value, refers to ±5%, or ±10%, of the numerical value.

In line with the above, the present application relates to ultrasonic treatment systems for intraoperatively disrupting and removing biological materials, such as microbial biofilms, from surfaces, such as implant surfaces and adjacent tissues, during orthopedic surgery and/or other procedures. Although described with reference to orthopedic applications, the disclosed systems can be utilized in a wide range of medical and non-medical applications, including but not limited to dental procedures, wound care, cardiovascular implants, endoscopic procedures, and treatment of non-biological surfaces. The disclosed systems may further be utilized for debridement of tissue, cleaning of implantable or non-implantable devices, and removal of adherent material from surfaces in both in vivo and ex vivo environments. The systems described herein may be used to remove material adhered to a wide variety of surfaces, including metallic, polymeric, ceramic, or composite surfaces. In some embodiments, the adhered material may include biological material, inorganic deposits, or combinations thereof.

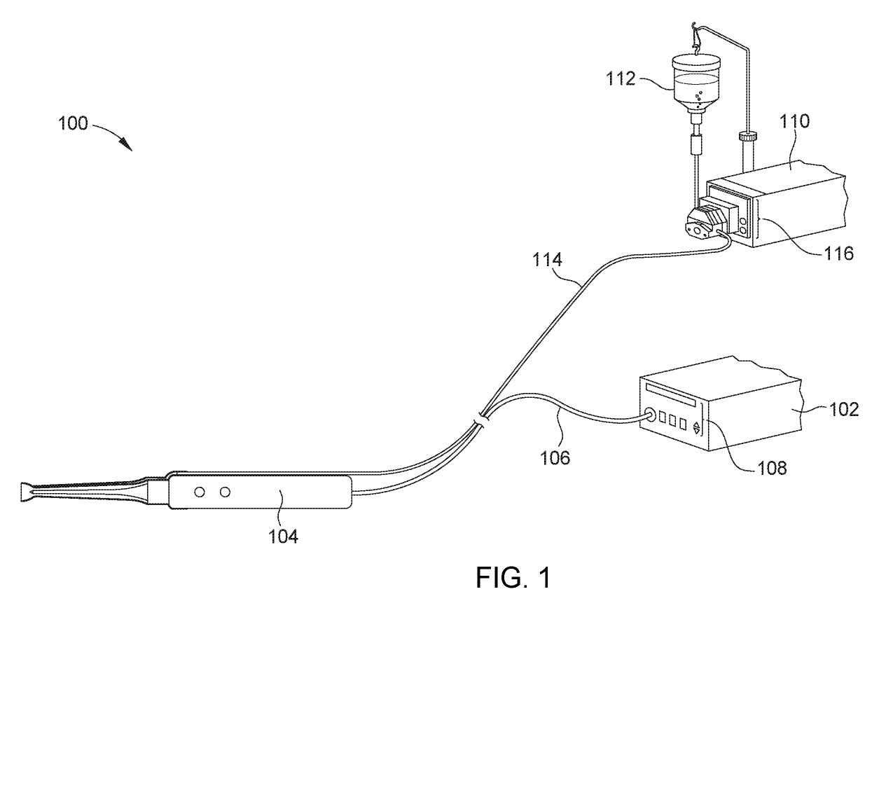

FIG. 1 is a schematic view of an example ultrasonic treatment system 100 for use during orthopedic and/or other surgical procedures, according to at least some embodiments of the present disclosure.

The ultrasonic treatment system 100 includes a control unit 102 that is configured to supply electrical power and control signals to a probe 104. The electrical power and control signals supplied by the control unit 102 are transmitted to the probe 104 via a flexible, wired electrical connection 106, which can be optionally shielded to reduce interference. In at least some embodiments, the control unit 102 includes a control interface 108 that allows a user (e.g., a surgeon or assistant) to activate and modulate system parameters for the ultrasonic treatment system 100, including the probe 104. In such embodiments, the control interface 108 can include one or more of a touchscreen or membrane switch panel, dial or button-based controls, wireless or wired foot pedal actuators, and/or voice command interfaces. Using the control interface 108, the user may selectively control activation or deactivation of ultrasonic energy, ultrasonic power output level, pulse duration, duty cycles, repetition rate, total treatment time, and/or the like to accommodate different surgical indications, circumstances, or tissue types. In at least some embodiments, the control unit 102 can have certain integrated safety features, such as an automatic shutoff, impedance sensing, preset treatment profiles optimized for specific surgical scenarios, and/or the like.

In some embodiments, the control unit 102 is configured to adjust one or more operating parameters, including ultrasonic frequency, amplitude, duty cycle, and fluid flow rate, to maintain a desired cavitation condition within the confined fluid volume. In some embodiments, the ultrasonic treatment system 100 includes one or more sensors configured to detect acoustic response, impedance, pressure, or fluid characteristics, and the control unit 102 is configured to adjust operation based on the detected parameters. Such sensors can be disposed on or within, for example, the probe 104.

The ultrasonic treatment system 100 further includes a pump 110 configured to drive controlled flow of irrigation fluid from a fluid source 112 via flexible tubing 114. In at least some embodiments, the flexible tubing 114 is fluidly coupled to the probe 104 to facilitate delivery of the irrigation fluid from the fluid source 112 to a treatment site. Suitable types of irrigation fluid can include saline, buffered solutions, sterile water, and antimicrobial or antiseptic solutions, which can be provided in a fluid bag. In at least some embodiments, the pump 110 is a peristaltic, roller, or diaphragm pump, selected for precision and compatibility with sterile surgical environments. The pump 110 can be integrated into the control unit 102 or provided as a separate console, as shown in FIG. 1. Where the pump 110 is integrated with the control unit 102, the pump is controllable by the control interface 108; where the pump 110 is provided separately, the pump 110 can be controlled via a separate control interface 116. Controllable parameters of the pump 110 can include fluid flow rate, fluid pressure, and/or pulsed or continuous flow modes.

In some embodiments, fluid delivery, as driven by the pump 110, can be continuous, pulsed, or synchronized with ultrasonic energy delivery to influence cavitation dynamics. In some embodiments, variations in fluid flow rate are used to modulate cavitation intensity and removal efficiency.

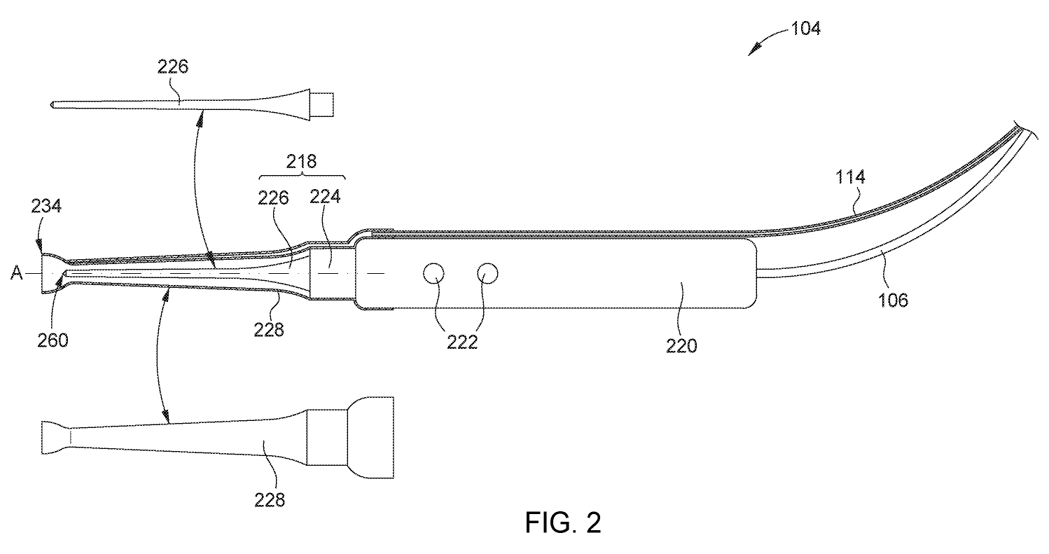

The probe 104, described in more detail with reference to FIG. 2, is configured to be held and manipulated by the user during a surgical procedure to deliver high-frequency (ultrasonic) mechanical vibrations to a treatment site for therapeutic purposes such as biofilm disruption and diagnostic purposes such as bacterial collection for identification. In conjunction with ultrasonic energy, the probe 104 can be further configured to direct the irrigation fluid driven by the pump 110 to the treatment site, which can provide a fluid medium to enhance energy transmission and induce cavitation to assist in loosening and removing biofilms with precision and minimal invasiveness.

FIG. 2 is a schematic and partially cross-sectional view of the probe 104 of FIG. 1, according to at least some embodiments of the present disclosure.

As shown, the probe 104 includes an ultrasonic assembly 218 that is mechanically and electrically coupled to a handpiece 220 configured to be held and manipulated by the user. The handpiece 220 provides ergonomic support and control, and may include integrated buttons, switches, or other toggles 222 to activate, deactivate, or modulate ultrasonic energy and/or fluid delivery during a procedure. In at least some embodiments, the handpiece 220 is shaped to accommodate a palm grip or pen-like grip, allowing for precise directional control of the probe 104. The handpiece 220 further houses and supports at least a proximal portion of the ultrasonic assembly 218. The handpiece 220 can be formed of biocompatible metals (e.g., stainless steel, titanium) or polymeric materials (e.g., PEEK) to ensure sterility and durability.

The ultrasonic assembly 218 includes a transducer 224 (e.g., an ultrasonic transducer), which includes an ultrasonic energy source configured to generate ultrasonic energy. In at least some embodiments, the transducer 224 includes a piezoelectric transducer, such as a piezoelectric stack or array that, when energized by electrical signals from the control unit 102, mechanically oscillates at ultrasonic frequencies. In other embodiments, the transducer 224 generates ultrasonic energy via other suitable mechanisms, such as by magnetostriction via a magnetostrictive transducer. Generally, the transducer 224 can produce ultrasonic energy in a frequency range of about 20 kHz to about 100 kHz or more, such as a frequency range of about 25 kHz to about 40 kHz. Frequencies within this range have been shown to effectively induce cavitation in fluid media, which can promote disruption of biofilms as further described below.

The ultrasonic assembly 218 further includes a horn 226 (e.g., an ultrasonic horn) coupled to the transducer 224 and extending distally from the handpiece 220. The horn 226 is configured to receive, amplify, and focus ultrasonic vibrations generated by the transducer 224. Amplification may be achieved through geometric features of the horn 226, such as tapered, stepped, or exponential profiles thereof. Generally, the horn 226 can be fabricated from high acoustic-efficiency materials such as titanium alloys, aluminum, or ceramic composites, which offer high mechanical strength and minimal energy loss.

A distal end 260 of the horn 226 can be configured with any suitable geometry to optimize the transmission characteristics of ultrasonic energy emitted therefrom. In some embodiments, the distal end 260 can be or include a surface that is planar, tapered, pointed, rounded, or angled, or can include combinations thereof. The specific geometry selected can be based on various factors including, but not limited to, a desired directionality, intensity distribution, focus, or coupling efficiency of ultrasonic waves into a transmission medium or treatment site. For example, a planar geometry can be suitable for broad, uniform energy distribution, while a tapered or pointed geometry can increase directional focus or penetration depth. Further, an angled or curved distal end 260, and/or a tapered or pointed distal end 260, can be utilized to direct ultrasonic energy along a path non-axial with a longitudinal axis A (described below) of the probe 104 to reach treatment sites that are, e.g., physically obstructed or in confined spaces.

In at least some embodiments, the horn 226 is removable and interchangeable, allowing different horn geometries or sizes to be selected based on the specific surgical procedure or treatment site. For example, a surgeon may select a horn 226 with a narrower tip for accessing confined anatomical spaces, or a broader horn for treating larger surface areas, enabling optimized energy delivery tailored to the clinical application.

At least a distal portion of the ultrasonic assembly 218, including the horn 226, is configured to be enclosed or encased by a sheath 228. The sheath 228, which is described in further detail below, facilitates controlled application of ultrasonic energy to a treatment site, and in at least some embodiments, also includes one or more fluid ports and/or channels for directing irrigation fluid to the treatment site. The irrigation fluid, typically sterile saline or an antimicrobial solution as noted above, serves as a fluid transmission medium for ultrasonic energy and enhances cavitation at the site of application.

The sheath 228 can be a component of the probe 104, or a separate component configured to attach to the probe 104. In at least some embodiments, the sheath 228, like the horn 226, is removable and interchangeable. This enables the user to select a sheath configuration that corresponds to the selected horn 226 and/or the clinical requirements of the surgical procedure. Different sheaths 228 may vary in length, diameter, flexibility, geometry, and/or fluid delivery features to accommodate various horn geometries and facilitate desired ultrasonic energy transmission and fluid flow at diverse treatment sites.

Generally, the components of the probe 104 together form an elongate member that has a generally cylindrical or tapered geometry, with the longitudinal axis A extending between the handpiece 220 and the distal end 260 of the horn 226 and/or a distal end 234 of the sheath 228.

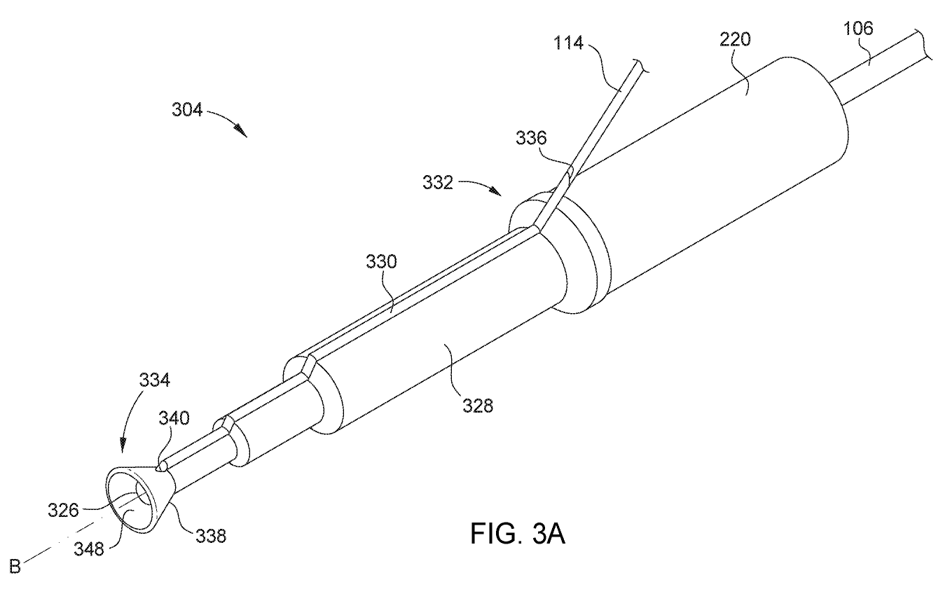

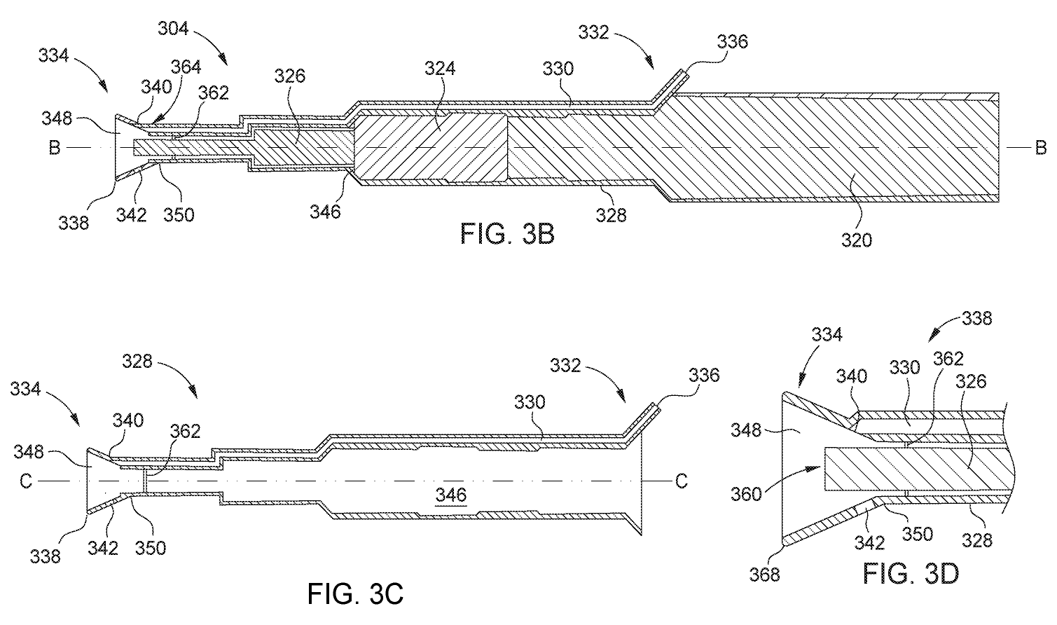

FIG. 3A is a more detailed perspective view of an example probe 304 of the ultrasonic treatment system of FIG. 1, with the example sheath 328 attached thereto, according to at least some embodiments of the present disclosure. FIG. 3B is a schematic cross-sectional view of the probe 304 with the sheath 328, according to at least some embodiments of the present disclosure. FIG. 3C is a cross-sectional view of the sheath 328, according to at least some embodiments of the present disclosure. FIG. 3D is an enlarged cross-sectional view of a sheath head 338 of the sheath 328, according to at least some embodiments of the present disclosure. For clarity, FIGS. 3A-3D are herein described together. Note that, in at least some embodiments, the probe 304 and/or sheath 328 are representative of the probe 104 and/or sheath 228, respectively.

As shown, the sheath 328 is configured to fit over one or more other components at a distal portion of the probe 304, and more particularly, over at least a transducer 324 and/or a horn 326 of the probe 304, which can be substantially similar to the transducer 224 and horn 226, respectively. A body 344 of the sheath 328 defines an internal cavity 346 dimensioned to receive and enclose at least the transducer 324 and/or horn 326 coupled thereto, and in some embodiments, can also accommodate one or more additional components of the probe 304, such as electrical components, vibration dampeners, fluid delivery conduits, and/or the like. The sheath 328 serves, in part, to isolate and protect the enclosed components, and to aid in guiding or positioning the probe 304 during a surgical procedure. In certain embodiments, the body 344 can be generally conical or cylindrical in shape.

The sheath 328 is generally formed from one or more biocompatible and surgical-grade materials, such as medical-grade polymers or elastomers, including but not limited to polyethylene, polypropylene, polyurethane, silicone, or other thermoplastic or thermoset elastomers. In some embodiments, the sheath 328 can be formed from a transparent or translucent material, allowing the user to view internal components of the probe 304 during use. In some embodiments, the sheath 328 can be partially or wholly rigid; in others, the sheath 328 can include flexible or elastomeric sections, such as at a distal end 334 of the sheath 328, to accommodate probe motion or anatomical constraints. An exterior profile of the body 344 of the sheath 328 can be generally tapered (e.g., conical or frustum-shaped) or stepped from a proximal end 332 of the sheath 328 to the distal end 334, facilitating insertion into a treatment site and allowing for ergonomic handling by the user.

In certain embodiments, the sheath 328 can be provided in a sterile, single-use form, while in other embodiments it may be reusable and configured for sterilization by autoclave, chemical treatment, or other suitable methods. The sheath 328 may also be provided in various sizes or shapes to match specific surgical procedures or anatomical locations.

In some embodiments, the body 344 of the sheath 328 attaches to a handpiece 320 of the probe 304. In some embodiments, the sheath 328 attaches to one or more other components of the probe 304. To secure the sheath 328 to the probe 304, the sheath 328 and/or corresponding component(s) the probe 304 can incorporate any suitable attachment mechanism that provides a secure yet releasable connection therebetween. This attachment mechanism can include a threaded, interference-fit, press-fit, or snap-fit coupling between the body 344 of the sheath 328 and the other component(s) of the probe 304, such as for example, the handpiece 320. In some embodiments, to enhance retention and alignment during use, the body 344 of the sheath 328 and/or corresponding component(s) of the probe 304 can include locking tabs, grooves, or detents. The releasable nature of the sheath 328 permits it to be readily removed and/or exchanged, for example, to facilitate desired ultrasonic energy transmission and fluid flow at diverse treatment sites, to accommodate different probe 304 geometries, and/or to replace the sheath 328 between procedures.

In some embodiments, the sheath 328 includes a fluid conduit 330 extending from the proximal end 332 to the distal end 334. In some embodiments, fluid delivery may occur without a conduit extending along the body 344, as described further below. The fluid conduit 330 can include a tube-like component that extends along an exterior or interior (e.g., within the internal cavity 346) of the body 344 of the sheath 328. At the proximal end 332, the fluid conduit 330 is in fluid communication with a port 336 adapted to receive or attach to flexible fluid tubing, such as tubing 114 described above. The port 336 therefore allows for connection to an external fluid source and/or pump, such as the fluid source 112 and pump 110, enabling controlled delivery of fluid into/through the fluid conduit 330. In various embodiments, the port 336 can be configured to interface with the tubing 114 using a variety of connection types. These can include a barbed or ribbed connector for frictional or press-fit engagement, a threaded luer-lock fitting for secure, twist-lock attachment, or a quick-connect or snap-fit mechanism to facilitate rapid connection and disconnection. In some embodiments, the port 336 can include an integrated clamp, collet, or compression fitting to enhance sealing and prevent dislodgement during use.

In some embodiments, the port 336 is oriented and configured to receive the tubing 114 along an axis (e.g., from a direction) that is generally parallel to an axis B of the probe 304. In such embodiments, the tubing 114 can extend along or adjacent to an outer surface of the handpiece 320 when connected, which can streamline the profile of the probe 304. In other embodiments, the port 336 is oriented at an angle that is non-parallel to the axis B, such as at an obtuse or normal angle relative to the axis B. In such embodiments, the tubing 114 extends away from the handpiece 320 when connected, creating a gap between the tubing 114 and the handpiece 320. This allows a user to comfortably grip the handpiece 320 without gripping or compressing the tubing 114, thereby maintaining unobstructed fluid flow through the tubing 114 during use.

At the distal end 334 of the sheath 328, the fluid conduit 330 terminates at or within a sheath head 338 that is integrally formed with, or fixedly or removably attachable to, the body 344. Where the sheath head 338 is removably attachable to the body 344, the sheath head 338 can be secured to the body 344 via any suitable attachment mechanism that provides a secure yet releasable connection therebetween. This attachment mechanism can include a threaded, interference-fit, press-fit, or snap-fit coupling between the sheath head 338 and the body 344 of the sheath 328. In some embodiments, to enhance retention and alignment during use, the sheath head 338 and/or the body 344 can include locking tabs, grooves, or detents. A removably attachable sheath head 338 permits the sheath head 338 to be readily removed and/or exchanged with other sheath heads during or between uses.

In some embodiments, as shown in FIGS. 3A-3C, the sheath head 338 is conical in shape to provide a sufficient internal volume 348 for fluid accumulation and ultrasonic energy propagation. More particularly, the sheath head 338 is a right circular cone with a circular base perpendicular to a central axis of the cone. However, other geometries for the sheath head 338, such as bulbous, cup-like, cylindrical, or flattened dome shapes, are also contemplated depending on the anatomical site and intended clinical application, as further described below. The sheath head 338 is configured to be pressed against a treatment site, such as a prosthetic implant, forming a fluid-tight seal thereagainst to isolate the treatment site from the surrounding tissues/environment.

A fluid inlet 340 is formed in a circumferential sidewall of the sheath head 338 and is in fluid communication with a distal end 364 of the fluid conduit 330 to allow irrigation fluid to flow into the internal volume 348 of the sheath head 338. A fluid outlet 342 is also formed in the sidewall of the sheath head 338, spaced from the fluid inlet 340 to allow passive egress of the irrigation fluid. In some embodiments, the fluid inlet 340 and the fluid outlet 342 are disposed 180° from each other along a circumference of the sidewall of the sheath head 338. In some embodiments, the fluid inlet 340 and the fluid outlet 342 are disposed less than 180° from each other along the circumference of the sidewall of the sheath head 338. In some embodiments, the fluid inlet 340 is disposed distal relative to the fluid outlet 342 along an axis C (FIG. 3B) of the sheath 328, which can be generally parallel to the axis B of the probe 304. In some embodiments, the fluid outlet 342 is disposed distal relative to the fluid inlet 340 along the axis C. In some embodiments, the fluid outlet 342 and the fluid inlet 340 are aligned along the axis C.

During operation, irrigation fluid is actively introduced into the internal volume 348 of the sheath head 338 via the fluid inlet 340. The relative positioning of the fluid inlet 340 and the fluid outlet 342 ensures that the sheath head 338 fills with irrigation fluid before the fluid begins to passively exit through the fluid outlet 342, thereby maintaining a fluid medium within the internal volume 348 of the sheath head 338 during use. This fluid medium serves as a transmission environment through which ultrasonic energy from the probe 304 can travel. The fluid medium can therefore be referred to as a “fluid transmission medium” in some embodiments herein. The interaction of ultrasonic energy with the fluid can generate cavitation effects, which assists in disrupting bacterial biofilms (or otherwise treating the targeted site).

In some embodiments, fluid is allowed to passively egress from the sheath head 338 without the application of active suction. In contrast to systems that actively evacuate fluid from a treatment region, passive fluid retention within the sheath head 338 can maintain a relatively stable fluid environment conducive to cavitation. In some scenarios, active suction may induce increased fluid flow velocity within the treatment region, which can disrupt formation and stability of cavitation bubbles and reduce cavitation efficacy. Additionally, application of suction in some scenarios may reduce local ambient pressure within the fluid volume, which can alter cavitation dynamics and reduce effective energy transfer at the treatment surface. Accordingly, in some embodiments, the ultrasonic treatment system 100 is configured to minimize or avoid active suction during cavitation-based treatment to enhance biofilm disruption efficiency.

Although described above as facilitating passive egress of irrigation fluid from the sheath head 338, in some embodiments, the fluid outlet 342 can be in fluid communication with a fluid conduit separate from, but similar to, the fluid conduit 330 to provide active fluid aspiration from the sheath head 338. In such embodiments, the separate fluid conduit can be configured to couple to a vacuum source of the ultrasonic treatment system 100 via flexible fluid tubing to facilitate active suction of irrigation fluid introduced into the sheath head 338 via the fluid inlet 340.

In some embodiments, the sheath 328 may omit the fluid conduit 330 extending along the body 344. In such embodiments, fluid may be delivered directly to the sheath head 338. For example, in some embodiments, the port 336 is arranged at or proximate to the sheath head 338, rather than at the proximal end 332 of the sheath 328. In such configurations, flexible tubing 114 may be directly coupled to the port 336 at the sheath head 338 to deliver fluid into the internal volume 348. The port 336 may be positioned at any suitable location along the sheath head 338, including at a distal portion, a proximal portion, or along a circumferential sidewall of the sheath head 338. In some embodiments, the port 336 is in direct fluid communication with the internal volume 348 of the sheath head 338, thereby enabling fluid to be introduced into the internal volume without passage through a conduit extending along the body 344. Such configurations may reduce structural complexity of the sheath 328, improve fluid delivery efficiency, and allow for more compact or flexible sheath designs. In some embodiments, the sheath 328 may include both the fluid conduit 330 and a port 336 located at or near the sheath head 338, thereby providing multiple fluid delivery pathways.

In some embodiments, the body 344 and/or the sheath head 338 are shaped and sized such that a distal end 360 of the horn 326 extends into the internal volume 348 of the sheath head 338. For example, in at least some embodiments, the distal end 360 of the horn 326 can extend into the sheath head 338 and distally beyond the fluid inlet 340 and/or the fluid outlet 342. Such configurations may allow ultrasonic energy to be directly focused into the fluid-filled internal volume 348, and/or beyond potentially turbulent fluid egress/ingress points in the sheath head 338, facilitating efficient transmission and cavitation at the treatment site. In other embodiments, the distal end 360 of the horn 326 may terminate proximal to the sheath head 338, with ultrasonic vibrations being directed through an opening between the body 344 and the sheath head 338.

In some embodiments, the body 344 and/or the sheath head 338 are shaped and sized such that the body 344 and/or the sheath head 338 functions as a spacer, establishing an optimal, or target, stand-off distance between the distal end 360 of the horn 326 and the treatment site when the sheath head 338 is pressed against tissue or a prosthetic surface. This predefined spacing is designed to facilitate efficient and consistent delivery of ultrasonic energy, optimizing the focal zone for interaction with the treatment site. By maintaining a controlled distance, the probe 304 enhances the effectiveness of ultrasonic energy in disrupting and removing at least portions of biofilms, or for performing other therapeutic or diagnostic actions, while minimizing unintended energy dissipation or damage to surrounding structures.

In some embodiments, one or more operational parameters of the ultrasonic treatment system 100 are selected or adjusted to control characteristics of the cavitation generated within the confined fluid volume. Such parameters may include ultrasonic frequency, amplitude, duty cycle, pulse duration, fluid type, fluid flow rate, and stand-off distance. In some embodiments, these parameters are selected to optimize cavitation intensity, spatial distribution, or stability within the confined fluid volume. In some embodiments, different parameter sets are used for different treatment objectives, including biofilm disruption, tissue debridement, or diagnostic sampling.

In some embodiments, the stand-off distance, and thus, the distance between the distal end 360 of the horn 326 and a distal end 334 of the sheath 328 and/or sheath head 338, can range from about 3 mm to about 1 cm, such as from about 4 mm to about 1 cm, or from about 5 mm to about 1 cm, or from about 6 mm to about 1 cm, or from about 7 mm to about 1 cm, or from about 8 mm to about 1 cm, or from about 9 mm to about 1 cm, or from about 3 mm to about 9 mm, or from about 3 mm to about 8 mm, or from about 3 mm to about 7 mm, or from about 3 mm to about 6 mm, or from about 3 mm to about 5 mm, or from about 3 mm to about 4 mm, or from about 4 mm to about 9 mm, or from about 5 mm to about 8 mm, or from about 6 mm to about 7 mm, or similar range. In some embodiments, the stand-off distance can be less than 3mm, such as about 2 mm or less or about 1 mm or less. In some embodiments, the stand-off distance can be more than 1 cm, such as about 2 cm or more or about 3 cm or more.

In some embodiments, the sheath 328 is monolithic in form, meaning that the body 344, sheath head 338, and/or the fluid conduit 330 are all formed of the same continuous material, such as a molded medical-grade polymer. In other embodiments, the sheath head 338, and/or the fluid conduit 330 can be formed from different materials than the body 344, and/or can be removably attachable to the body 344. For example, in some embodiments, the sheath head 338 can be constructed of a more flexible material relative to other, more rigid portions of the sheath 328, such as the body 344. In some embodiments, a junction 350 between the body 344 and the sheath head 338 is configured to be more flexible relative to other, more rigid portions of the sheath 328. This multi-material and/or modular construction can allow for enhanced performance tailored to specific procedural requirements. For example, localized flexibility can allow for limited angular manipulation of the probe 304 while the sheath head 338 is pressed against a treatment site, thereby enabling controlled adjustment of the direction or angle at which ultrasonic energy is delivered through the fluid medium within the sheath head 338. Additionally, increased flexibility of the sheath head 338 can optimize sealing characteristics thereof.

In some embodiments, the sheath head 338 includes a lip 368 having a rounded, curved, or beveled surface. This geometry facilitates improved sealing engagement when the sheath head 338 is pressed against a treatment site, such as tissue or a prosthetic implant. Additionally, the curved lip 368 allows the sheath head 338 to be more easily slid or repositioned along the surface of the treatment site while maintaining contact, thereby enabling fine adjustment of the probe position during a procedure without damaging or scraping the tissue or implant.

In some embodiments, the sheath 328 includes a membrane 362 that extends laterally across a portion of the internal cavity 346, such as at or near the distal end 334 of the sheath 328. The membrane 362 is configured to maintain a protective fluid barrier within the sheath 328 and can be formed of any one or more suitable medical-grade plastics, elastomers, or similar flexible materials that provide durability and biocompatibility. In some embodiments, the membrane 362 is adapted to be punctured by the horn 326 of the probe 304 during attachment of the sheath 328 onto the probe 304, thereby allowing at least a portion of the horn 326 to pass through the membrane 362 and toward the sheath head 338. In some embodiments, the membrane 362 includes a preformed aperture, slit, or valve-like opening that is dimensioned to receive the horn 326 therethrough without the need for puncture.

The membrane 362 is configured to form a seal around the horn 326 once inserted therein, thereby preventing backflow or leakage of fluids (e.g., irrigation fluids) proximally through the sheath 328 during use. This sealing function is particularly advantageous during procedures wherein the probe 304 is manipulated at steep or extreme angles relative to, e.g., a surface of a treatment site. By maintaining a secure fluid barrier, the membrane 362 helps protect electronic and other components of the probe 304 from contamination or damage due to fluid ingress.

In use, the sheath head 338 is pressed against a treatment site to form a fluid-sealed interface, thereby defining a confined fluid volume. Ultrasonic energy transmitted through the fluid medium generates cavitation within the confined volume, which mechanically disrupts and detaches biofilm from the treatment site. The confined nature of the fluid volume increases cavitation intensity relative to unconfined fluid environments. In some embodiments, the confined fluid volume allows spatial confinement of cavitation effects to a region directly adjacent to the treatment site, thereby limiting propagation of cavitation into surrounding areas. This spatial control can reduce unintended effects on adjacent tissue while maintaining effective disruption at the target surface.

In some embodiments, the effectiveness of cavitation may vary depending on factors such as fluid composition, surface geometry, and degree of sealing between the sheath head 338 and the treatment site. The ultrasonic treatment system 100 can be operated under varying conditions to compensate for such factors, including adjusting fluid flow, repositioning the sheath head, or modifying ultrasonic parameters. In some embodiments, partial sealing between the sheath head 338 and the treatment site may still provide sufficient confinement to enhance cavitation relative to unconfined fluid environments. For example, in some embodiments, the sheath head 338 is configured such that complete sealing is not required to achieve enhanced cavitation effects, and partial confinement of the fluid is sufficient to increase cavitation intensity relative to fully unconfined systems. This can allow effective operation even under dynamic surgical conditions where perfect sealing cannot be maintained.

In some embodiments, the ultrasonic treatment system 100 may be used in combination with additional treatment modalities, including chemical agents, antimicrobial solutions, or mechanical agitation. For example, antimicrobial agents may be introduced into the confined fluid volume to enhance treatment effectiveness. In some embodiments, ultrasonic cavitation may improve penetration of such agents into biofilm structures.

In some embodiments, ultrasonic energy is delivered in a pulsed or intermittent manner. In some embodiments, fluid delivery is synchronized with ultrasonic energy delivery. In some embodiments, different phases of operation are employed, including an initial disruption phase followed by a flushing or removal phase.

In some embodiments, the geometry of the horn 326, the sheath head 338, or both, is configured to direct or vector ultrasonic energy and resulting cavitation effects toward a desired region of a treatment surface. Such vectoring may be achieved through selection of horn geometry, including angled, tapered, or curved distal ends, in combination with sheath head geometries that define an asymmetric or directionally biased confined fluid volume. In some embodiments, the resulting cavitation field is non-uniform and preferentially directed toward non-orthogonal or difficult-to-access surfaces. This enables effective treatment of angled, recessed, or partially obstructed surfaces that may not be effectively addressed by axially directed ultrasonic systems.

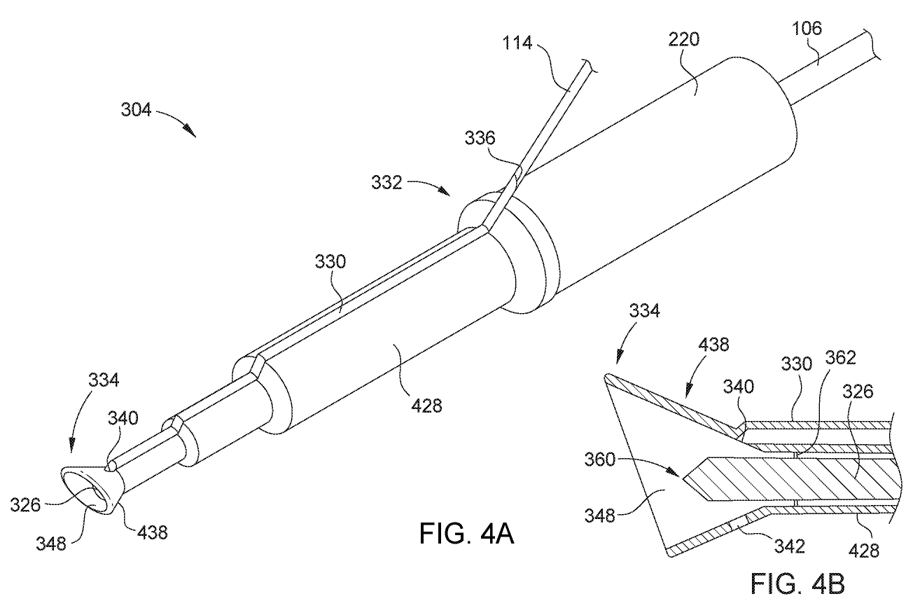

FIG. 4A is a perspective view of the probe 304 with another example sheath 428, according to at least some embodiments of the present disclosure. Note that, in at least some embodiments, the sheath 428 is representative of the sheath 228. FIG. 4B is an enlarged cross-sectional view of a sheath head 438 of the sheath 428, according to at least some embodiments of the present disclosure. For clarity, FIGS. 4A and 4B are herein described together.

In the example of FIGS. 4A and 4B, the sheath 428 is generally similar in structure and function to the previously described sheath 328. Accordingly, similar features are denoted by corresponding reference numerals shown in FIGS. 3A–3D.

The key variation between the sheath 428 and the sheath 328 is the geometry of the sheath head 438. As shown, in some embodiments, the sheath head 438 is shaped as an oblique cone, wherein a central axis of the cone is not perpendicular to its base, resulting in a shorter portion and a longer portion along the circumference of the cone. That is, an apex of the cone is offset relative to the central axis of the cone, resulting in a slanted or tilted conical geometry. In some embodiments, the sheath head 438 is a right circular cone but includes an angled, elliptical base, which similarly produces an asymmetric height profile. In either case, the asymmetry creates distinct regions along the circumference of the sheath head 438 that may be advantageous for targeted access or positioning during certain procedures. In some embodiments, these configurations enable the sheath head 438 to engage a treatment site at an oblique angle, which can be beneficial when maneuvering around anatomical structures or accessing angled surfaces of prosthetic implants. For example, when treating prostheses with non-orthogonal surfaces or hard-to-reach periprosthetic regions, an oblique or slanted sheath head 438 can facilitate improved contact and sealing against the treatment site for more effective formation of a fluid transmission environment through which ultrasonic energy from the probe 304 can travel.

In certain embodiments, the sheath 428 having the asymmetrical sheath head 438 can be utilized in combination with the probe 304 having the horn 326 with a tapered, pointed, angled, or curved distal end 360. In the example of FIG. 4B, a pointed distal end 360 is shown. As noted above, a tapered, pointed, angled, or curved distal end 360 can be utilized to direct ultrasonic energy along a path non-axial with the probe 304. Thus, combining the angled horn 326 with the asymmetrical sheath head 438 can facilitate improved targeting of treatment sites being treated at oblique angles, such as treatment sites that are, e.g., physically obstructed, angled, or in confined spaces.

Similar to the sheath head 338, the fluid inlet 340 and the fluid outlet 342 are each formed in the sidewall of the sheath head 438 and spaced apart from each other to allow passive egress of the irrigation fluid. In some embodiments, the fluid inlet 340 is positioned adjacent the shorter portion of the sheath head 438, while the fluid outlet 342 is positioned adjacent the longer portion. In other embodiments, this arrangement can be reversed. Alternatively, one or both of the fluid inlet 340 and the fluid outlet 342 can be located circumferentially between the shorter and longer portions of the sheath head 438. Strategic placement of these holes can promote desired directional flow of irrigation fluid and allow passage of ultrasonic energy while maintaining a stable fluid medium between the horn 326 and the treatment site.

During operation, irrigation fluid is actively introduced into the internal volume 348 of the sheath head 338 via the fluid inlet 340. The relative positioning of the fluid inlet 340 and the fluid outlet 342 ensures that the sheath head 338 fills with irrigation fluid before the fluid begins to exit through the fluid outlet 342, thereby maintaining a fluid medium within the internal volume 348 of the sheath head 338 during use. This fluid medium serves as a transmission environment through which ultrasonic energy from the probe 304 can travel. The interaction of ultrasonic energy with the fluid can generate cavitation effects, which assists in disrupting bacterial biofilms (or otherwise treating the targeted site) for treatment and/or diagnostic purposes.

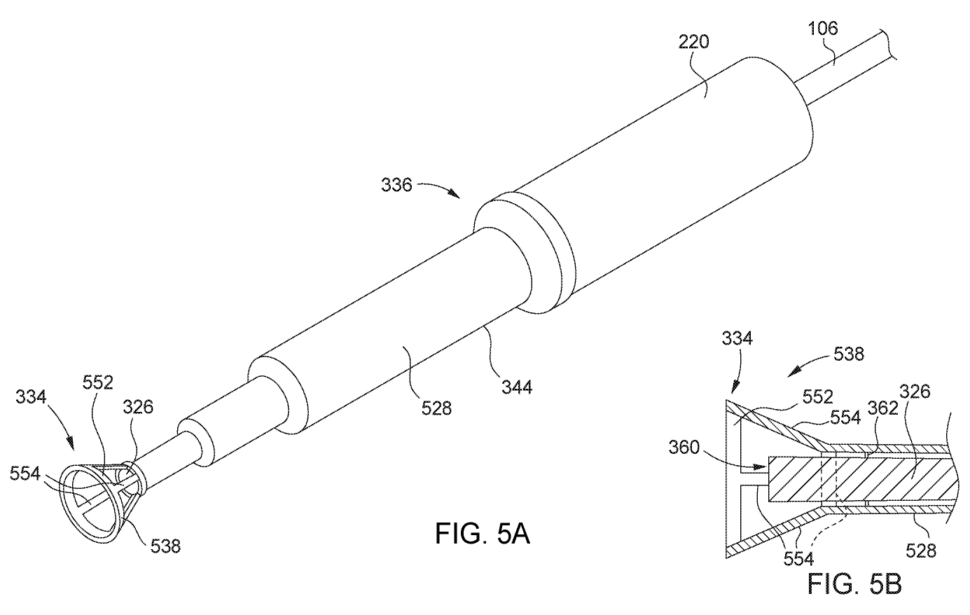

FIG. 5A is a perspective view of the probe 304 with another example sheath 528, according to at least some embodiments of the present disclosure. Note that, in at least some embodiments, the sheath 528 is representative of the sheath 228. FIG. 5B is an enlarged cross-sectional view of a sheath head 538 of the sheath 528, according to at least some embodiments of the present disclosure. For clarity, FIGS. 5A and 5B are herein described together.

The sheath 528 is generally similar in structure and function to the previously described sheath 328 and/or 428. Thus, similar features are denoted by corresponding reference numerals shown in FIGS. 3A–3D and/ or 4A and 4B.

The key variations between the sheath 528 and the sheath 328 and/or 428 are the configuration of the sheath head 538 and the omission of a fluid conduit (e.g., 330 or 430). In the examples of FIGS. 5A and 5B, the sheath head 538 resembles a right conical basket formed by one or more rings 552 circumscribing one or more stakes 554 extending distally from the body 344. However, the sheath head 538 is not limited to a conical configuration. Rather, the three-dimensional arrangement of the rings 552 and stakes 554 can define a wide variety of geometries, including oblique conical, frustoconical, cylindrical, spherical, hemispherical, domed, cuboid, polygonal, or other suitable forms. In some embodiments, the rings 552 can define circular, elliptical, or polygonal profiles, and the stakes 554 can be arranged linearly, radially, helically, or in other patterns to define the overall shape and structural characteristics of the sheath head 538. Accordingly, the term “basket” as used herein refers to an open-frame or skeletal structure defined by structural members (e.g., rings 552 and/or stakes 554), and does not require any particular geometric form, symmetry, or taper.

Unlike the previous enclosed sheath head designs, the basket-style sheath head 538 is open, allowing fluid to pass through interstices between the rings 552 and/or stakes 554 and across a path of ultrasonic energy emitted by the horn 326 of the probe 304. In some embodiments, the geometry, spacing, thickness, and arrangement of the rings 552 and/or stakes 554 are selected such that fluid can pass relatively freely through the sheath head 538, thereby permitting substantial fluid exchange with the surrounding treatment environment. In other embodiments, the geometry and/or spacing of the rings 552 and/or stakes 554 are selected to restrict bulk fluid flow and limit fluid exchange, thereby providing at least partial confinement of fluid, cavitation bubbles, and detached material within a localized treatment region adjacent to the treatment site. Configurations that permit relatively free fluid passage can be particularly beneficial when a treatment site is already submerged in fluid, such as saline, irrigation fluid, or bodily fluid, which can serve as the transmission medium for ultrasonic energy delivered by the probe 304. In such embodiments, free fluid exchange can facilitate continuous replenishment of fluid, removal of debris, and thermal regulation during operation. In contrast, configurations that provide partial confinement can promote retention of fluid and suspended material within the localized treatment region, thereby increasing cavitation density, enhancing interaction between cavitation events and the treatment surface, and improving detachment and retention of biological material for removal and/or diagnostic analysis.

Accordingly, in some embodiments, active fluid flow (e.g., irrigation) through a fluid conduit of the sheath 528 may not be necessary, as the surrounding fluid of the treatment site can provide the necessary conditions for effective energy delivery. Thus, in at least some embodiments, the sheath 528 may not include a fluid conduit as described with previous sheaths. In such embodiments, the sheath head 538 may function primarily as a spacer, establishing an optimal stand-off distance between the distal end 360 of the horn 326 and the treatment site when the sheath head 538 is pressed against tissue or a prosthetic surface. In some embodiments, the sheath head 538 can be configured to operate across a spectrum between relatively unconfined and partially confined conditions, allowing a user to select or utilize a desired balance between fluid exchange and cavitation localization based on procedural requirements.

In some embodiments, the disclosed systems are utilized during a surgical procedure to treat a contaminated implant site. For example, during a debridement procedure, a surgeon may position the sheath head of a probe against a surface of an implant and introduce irrigation fluid to establish a confined fluid volume. Ultrasonic energy is then delivered into the confined fluid volume to generate cavitation, thereby disrupting biofilm adhered to the implant surface. The disrupted material may be removed via fluid flow or suction, and in some embodiments, collected for diagnostic evaluation. The procedure may be repeated across multiple regions of the treatment site without removal of the implant. In some embodiments, the sheath head can be repositioned across the treatment site in a scanning or sweeping motion to treat multiple regions. In some embodiments, overlapping treatment zones are applied to ensure complete coverage of the treatment surface.

In some embodiments, retention of fluid within the sheath head due to passive or limited outflow enables accumulation of dislodged biological material within the confined volume. This accumulation may increase the concentration of biological material available for collection relative to systems employing continuous suction or high-flow irrigation. As a result, the disclosed systems may improve the quality and reliability of diagnostic sampling from treated surfaces.

In some embodiments, the ultrasonic systems described herein are utilized to facilitate diagnostic sampling of a treatment site. For example, during operation, ultrasonic energy delivered into the confined fluid volume dislodges biological material adhered to a surface, including microbial biofilm, thereby suspending the material within the fluid. In some embodiments, ultrasonic cavitation enhances detachment of microorganisms from biofilm matrices, thereby increasing the concentration of biological material available for analysis relative to passive sampling techniques. This may improve sensitivity of downstream diagnostic assays.

The fluid containing the dislodged material can then be collected from the sheath head via passive outflow or active suction and transferred to a collection vessel. In some embodiments, the collected fluid is processed to isolate biological material for analysis. In some embodiments, the analysis includes identification of microbial species, detection of biofilm-associated organisms, quantification of microbial load, and/or determination of antimicrobial resistance. In some embodiments, the diagnostic sampling is performed in situ without removal of an implant or tissue from a patient. In some embodiments, the diagnostic sampling is performed intraoperatively to provide real-time or near-real-time information for clinical decision-making.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. The term “about,” when used in conjunction with a numerical value, refers to plus or minus 5%, or plus or minus 10%, of the numerical value. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

While various examples of the subject matter have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the disclosed subject matter, but instead as merely intended to highlight alternative or additional features that may or may not be utilized in a particular example of the subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosure to the specific examples and embodiments described herein, but rather to also cover all modification and alternatives coming with the true scope and spirit of the subject matter.

What is claimed is:

1. An ultrasonic system for removing a biofilm at a treatment site, comprising:
an ultrasonic probe, comprising:
an ultrasonic assembly, comprising:
a transducer configured to generate ultrasonic energy; and
a horn in operable communication with the transducer and configured to receive, amplify, and focus the ultrasonic energy generated by the transducer; and
a handpiece mechanically and electrically coupled to the ultrasonic assembly and configured to be held and manipulated by a user;
a sheath configured to be removably attached to the ultrasonic probe, the sheath comprising:
a body configured to define an internal cavity for receiving and enclosing at least the ultrasonic assembly; and
a sheath head coupled at a distal end of the body, the sheath head configured to perform at least one of:
function as a spacer to provide a target stand-off distance between a distal end of the horn and a treatment site when the sheath head is pressed against the treatment site; or
maintain fluid within an internal volume of the sheath head when the sheath head is pressed against the treatment site to provide a fluid transmission medium between the distal end of the horn and the treatment site.

2. The ultrasonic system of claim 1, wherein the sheath is formed of a medical-grade polymer or elastomer.

3. The ultrasonic system of claim 1, wherein the sheath is configured to removably couple to the handpiece via a threaded, interference-fit, press-fit, or snap-fit mechanism.

4. The ultrasonic system of claim 1, wherein the sheath further comprises a fluid conduit extending between a proximal end of the sheath and a distal end of the sheath, the fluid conduit for directing the fluid to the internal volume of the sheath head for forming the transmission medium.

5. The ultrasonic system of claim 4, wherein:
the fluid conduit fluidly couples to an inlet formed in a sidewall of the sheath head, the inlet configured to facilitate ingress of the fluid into the internal volume; and
the sheath head further comprises an outlet formed in the sidewall, the outlet configured to facilitate egress of the fluid out of the internal volume.

6. The ultrasonic system of claim 5, wherein the ingress is active and the egress is passive.

7. The ultrasonic system of claim 1, wherein the sheath head comprises a conical, bulbous, cup-like, cylindrical, or a flattened dome geometry.

8. The ultrasonic system of claim 1, wherein the sheath head comprises a lip, the lip comprising a rounded surface to facilitate sealing engagement with the treatment site.

9. The ultrasonic system of claim 1, wherein the sheath head comprises an open-frame geometry for enabling fluid to freely pass through the sheath head during use.

10. The ultrasonic system of claim 1, further comprising:
a membrane extending across a portion of the internal cavity and configured to form a seal around the horn when the sheath is attached to the ultrasonic probe, the seal preventing backflow of the fluid through the sheath.

11. A sheath for an ultrasonic probe, comprising:
a body configured to removably couple to an ultrasonic probe to enclose at least a horn of the ultrasonic probe; and
a sheath head coupled to a distal end of the body, the sheath head configured to perform at least one of:
function as a spacer to provide a target stand-off distance between a distal end of the horn and a treatment site when the sheath head is pressed against the treatment site; or
maintain a fluid within an internal volume of the sheath head when the sheath head is pressed against the treatment site to provide a fluid transmission medium between the distal end of the horn and the treatment site.

12. The sheath of claim 11, wherein the sheath head is configured to removably couple to the body via a threaded, interference-fit, press-fit, or snap-fit mechanism.

13. The sheath of claim 11, wherein the body is configured to removably couple to a handpiece of the ultrasonic probe via a threaded, interference-fit, press-fit, or snap-fit mechanism.

14. The sheath of claim 11, wherein the sheath further comprises a fluid conduit extending between a proximal end of the sheath and a distal end of the sheath, the fluid conduit for directing the fluid to the internal volume of the sheath head for forming the transmission medium.

15. The sheath of claim 14, wherein:
the fluid conduit fluidly couples to an inlet formed in a sidewall of the sheath head, the inlet configured to facilitate ingress of the fluid into the internal volume; and
the sheath head further comprises an outlet formed in the sidewall, the outlet configured to facilitate egress of the fluid out of the internal volume.

16. The sheath of claim 11, wherein the sheath head comprises a conical, bulbous, cup-like, cylindrical, or a flattened dome geometry.

17. The sheath of claim 11, wherein the sheath further comprises a lip, the lip comprising a rounded surface to facilitate sealing engagement with the treatment site.

18. The sheath of claim 11, wherein the sheath head comprises an open-frame geometry for enabling fluid to freely pass through the sheath head during use.

19. The sheath of claim 11, further comprising:
a membrane extending across a portion of the body and configured to form a seal around the horn when the sheath is attached to the ultrasonic probe, the seal preventing backflow of the fluid through the sheath.

20. A sheath for an ultrasonic probe, comprising:
a body configured to removably couple to an ultrasonic probe to enclose at least a horn of the ultrasonic probe;
a membrane extending across a portion of the body and configured to form a seal around the horn when the sheath is attached to the ultrasonic probe, the seal preventing backflow of fluids through the sheath; and
a sheath head coupled to a distal end of the body, the sheath head configured to:
function as a spacer to provide a target stand-off distance between a distal end of the horn and a treatment site when the sheath head is pressed against the treatment site; and
maintain a fluid within an internal volume of the sheath head when the sheath head is pressed against the treatment site to provide a fluid transmission medium between the distal end of the horn and the treatment site such that the internal volume defines a confined or partially confined fluid region adjacent to the treatment site in which ultrasonic energy delivered by the horn generates cavitation, wherein the confinement of the fluid region increases a density of cavitation events relative to an unconfined fluid environment.
